(19) United States
(12) Patent Application Publication
(10) Pub. No.: US 2020/0206010 A1
(43) Pub. Date: Jul. 2, 2020

(54) SPLINT AND OPERATION METHOD THEREOF

(71) Applicant: BOE TECHNOLOGY GROUP CO., LTD., Beijing (CN)

(72) Inventors: Zhihong DU, Beijing (CN); Wenbo LI, Beijing (CN)

(21) Appl. No.: 16/561,441

(22) Filed: Sep. 5, 2019

(30) Foreign Application Priority Data
Jan. 2, 2019 (CN) 201910002869.2

(51) Int. Cl. A61F 5/058

(52) U.S. Cl. CPC A61F 5/05825; A61F 5/05841

(57) ABSTRACT

A splint and an operation method thereof are provided. The splint includes a plurality of plates and a splint adjustment device. The splint adjustment device is connected to the plurality of plates and configured to allow the plurality of plates to move relatively close to each other or relatively away from each other. At least one of the plurality of plates comprises at least two split portions, and the at least two split portions are configured to be separated from each other or be combined with each other, so as to allow a space surrounded by the plurality of plates to be opened or closed.

### CROSS-REFERENCE

The present application claims priority of Chinese patent application No. 201910002869.2, filed on Jan. 2, 2019, the disclosure of which is incorporated herein by reference as part of the application.

### TECHNICAL FIELD

Embodiments of the present disclosure relate to a splint and an operation method thereof.

### BACKGROUND

In medical fields, plasters are commonly used to fix injured limbs of a patient to protect the injured limbs. For example, a plaster bandage and a plaster cast can be used to fix the injured limb of the patient. With the development of technologies, polymer bandages and splints are widely used in medical fields, gradually, and become a mainstream fixing method because of the advantages such as high strength, light weight, thin thickness, breathability, waterproofness, non-skin-allergy, ability to transmit X-rays, easy disassembly, and so on.

### SUMMARY

At least one embodiment of the present disclosure provides a splint, which comprises: a plurality of plates, and a splint adjustment device connected to the plurality of plates and configured to allow the plurality of plates to move relatively close to each other or relatively away from each other. At least one of the plurality of plates comprises at least two split portions, and the at least two split portions are configured to be separated from each other or be combined with each other, so as to allow a space surrounded by the plurality of plates to be opened or closed.

For example, in the splint provided by an embodiment of the present disclosure, the at least two split portions comprise a first split portion and a second split portion, the first split portion comprises a first connecting structure, and the second split portion comprises a second connecting structure, and the first connecting structure is configured to be combined with the second connecting structure, so as to fix a relative position between the first split portion and the second split portion.

For example, in the splint provided by an embodiment of the present disclosure, the first connecting structure comprises a buckle and the second connecting structure comprises a clamping slot, alternatively, the first connecting structure comprises a clamping slot and the second connecting structure comprises a buckle.

For example, in the splint provided by an embodiment of the present disclosure, a material of the first connecting structure or a material of the second connecting structure comprises a permanent magnet or an electromagnet.

For example, in the splint provided by an embodiment of the present disclosure, at least one of the plurality of plates comprising the at least two split portions comprises at least one group of adsorption components, the at least one group of adsorption components comprises a plurality of adsorption units, the plurality of adsorption units are respectively on each of the at least two split portions, and the adsorption components are configured to enable the at least two split portions to combine with each other.

For example, in the splint provided by an embodiment of the present disclosure, the adsorption units comprise permanent magnets, electromagnets, or Velcro straps.

For example, in the splint provided by an embodiment of the present disclosure, the adsorption units comprise permanent magnets or electromagnets, and the adsorption units are disposed at predetermined positions of the split portions according to a first predetermined pattern.

For example, in the splint provided by an embodiment of the present disclosure, the splint adjustment device comprises: a drawstring movably connected to the plurality of plates, and a drawstring drive device on one of the plurality of plates, connected to the drawstring and configured to be controllable to drive the drawstring, so that the drawstring is capable of being tightened or relaxed under a driving function of the drawstring drive device, to drive the plurality of plates to be relatively close to each other or relatively away from each other.

For example, the splint provided by an embodiment of the present disclosure further comprises a pulling force detection circuit, and the pulling force detection circuit is coupled to the drawstring and is configured to detect a pulling force applied to the drawstring.

For example, the splint provided by an embodiment of the present disclosure further comprises a display, and the display is on one of the plurality of plates, is in signal connection with the pulling force detection circuit, and is configured to display a pulling force detection result of the pulling force detection circuit.

For example, in the splint provided by an embodiment of the present disclosure, the display and the drawstring drive device are on a plate opposite to a plate comprising the at least two split portions.

For example, the splint provided by an embodiment of the present disclosure further comprises at least one magnetic component, and the at least one magnetic component is disposed on at least one plate of the plurality of plates according to a second predetermined pattern.

For example, in the splint provided by an embodiment of the present disclosure, the at least one magnetic component comprises a permanent magnet or an electromagnet.

At least one embodiment of the present disclosure further provides an operation method of the splint provided by any one of the embodiments of the present disclosure, and the operation method comprises: separating the at least two split portions from each other, and allowing a using object to get into a space surrounded by the plurality of plates through an opening formed by separating the at least two split portions; combining the at least two split portions with each other; and using the splint adjustment device to allow the plurality of plates to be relatively close to each other and to be attached to the using object.

At least one embodiment of the present disclosure further provides an operation method of the splint provided by any one of the embodiments of the present disclosure, and the operation method comprises: using the splint adjustment device to allow the plurality of plates to be relatively away from each other and away from a using object which is in a space surrounded by the plurality of plates; separating the at least two split portions from each other; and allowing the using object to move out of the space surrounded by the plurality of plates through an opening formed by separating the at least two split portions.

### BRIEF DESCRIPTION OF THE DRAWINGS

In order to demonstrate clearly technical solutions of the embodiments of the present disclosure, the accompanying drawings in relevant embodiments of the present disclosure will be introduced briefly. It is apparent that the drawings may only relate to some embodiments of the disclosure and not intended to limit the present disclosure.

FIG. 1 is a stereoscopic schematic diagram of a splint provided by some embodiments of the present disclosure;

FIG. 2 is a back view of the splint illustrated in FIG. 1;

FIG. 3 is a top view of the splint illustrated in FIG. 1;

FIG. 4 is a sectional schematic diagram of a second plate of the splint illustrated in FIG. 1;

FIG. 5 is a schematic diagram of an acupuncture point distribution of an arm of a human body;

FIG. 6 is a back view of another splint provided by some embodiments of the present disclosure;

FIG. 7 is a flow diagram of an operation method of a splint provided by some embodiments of the present disclosure; and

FIG. 8 is a flow diagram of another operation method of a splint provided by some embodiments of the present disclosure.

### DETAILED DESCRIPTION

In order to make objects, technical details and advantages of the embodiments of the disclosure apparent, the technical solutions of the embodiment will be described in a clearly and fully understandable way in connection with the drawings related to the embodiments of the disclosure. It is apparent that the described embodiments are just a part but not all of the embodiments of the disclosure. Based on the described embodiments herein, those skilled in the art may obtain other embodiment, without any creative work, which shall be within the scope of the disclosure.

Unless otherwise defined, all the technical and scientific terms used herein have the same meanings as commonly understood by one of ordinary skill in the art to which the present disclosure belongs. The terms, such as “first,” “second,” or the like, which are used in the description and the claims of the present disclosure, are not intended to indicate any sequence, amount or importance, but for distinguishing various components. The terms, such as “comprise/comprising,” “comprise/comprising,” or the like are intended to specify that the elements or the objects stated before these terms encompass the elements or the objects and equivalents thereof listed after these terms, but not preclude other elements or objects. The terms, such as “connect/connecting/connected,” “couple/coupling/coupled” or the like, are not limited to a physical connection or mechanical connection, but may comprise an electrical connection/coupling, directly or indirectly. The terms, “on,” “under,” “left,” “right,” or the like are only used to indicate relative position relationship, and when the position of the object which is described is changed, the relative position relationship may be changed accordingly.

In a case where a polymer bandage and a splint are used for fixing, it is required to wrap a plurality of splints separated from each other onto the injured limb of the patient by the polymer bandage. Therefore, it is difficult and spends long time to wrap the splints. Moreover, after the splints are fixed, problems such as looseness and displacement are easily to occur, which not only affect the fixing effect of the splints, but also may cause secondary injury to the patient and aggravate the patient's condition. Furthermore, the frequent disassembly of the splints increases the workload of the medical staff and reduces the work efficiency.

At least one embodiment of the present disclosure provides a splint and an operation method thereof. The splint can be opened or closed at a side surface, which is convenient for operation, and can enable the user to adjust and fix the splint easily and quickly, thereby reducing the workload of the user, improving the work efficiency, and improving the user's experience. The splint provided by at least some of the embodiments can also monitor the tightness degree.

In the following, the embodiments of the present disclosure are described in detail with reference to the accompanying drawings. It should be noted that the same reference numerals used in different drawings refer to the same components that are described.

At least one embodiment of the present disclosure provides a splint, and the splint comprises a plurality of plates and a splint adjustment device. The splint adjustment device is connected to the plurality of plates and configured to allow the plurality of plates to move relatively close to each other or relatively away from each other. At least one of the plurality of plates comprises at least two split portions, and the at least two split portions are configured to be separated from each other or be combined with each other, so as to allow a space surrounded by the plurality of plates to be opened or closed.

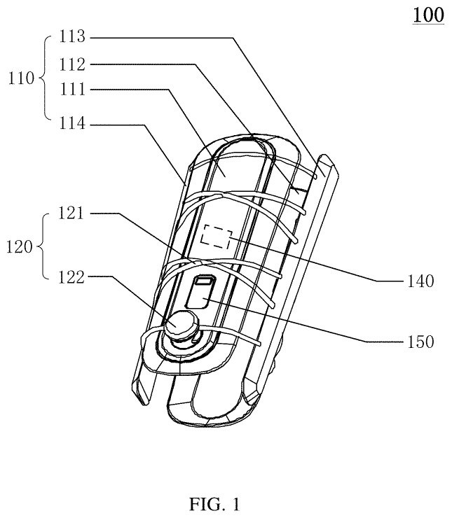

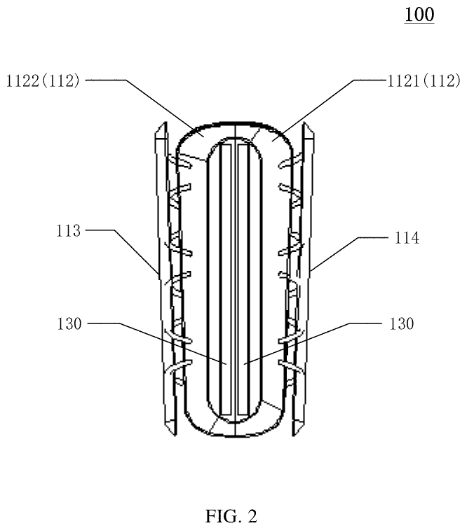

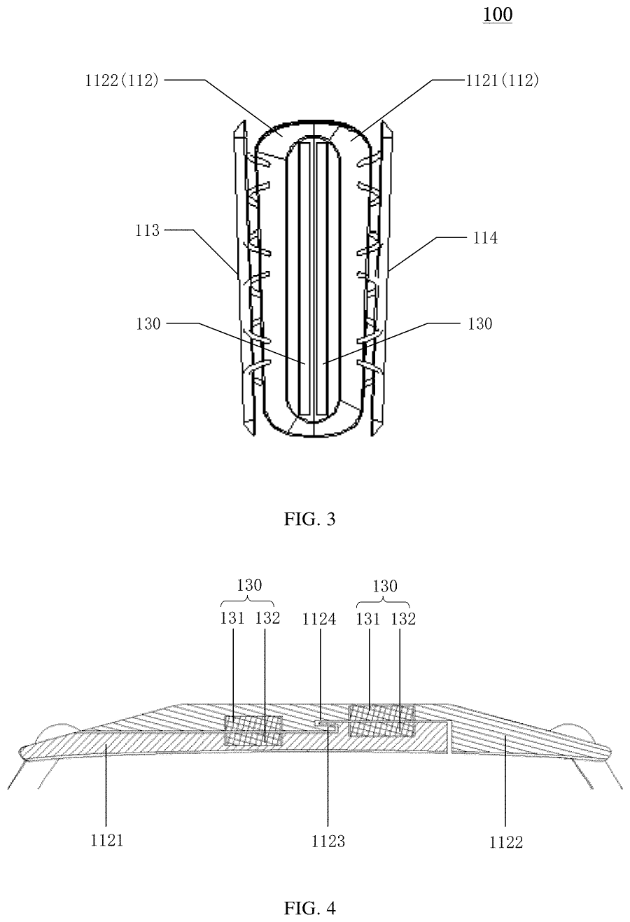

FIG. 1 is a stereoscopic schematic diagram of a splint provided by some embodiments of the present disclosure, FIG. 2 is a back view of the splint illustrated in FIG. 1, FIG. 3 is a top view of the splint illustrated in FIG. 1, and FIG. 4 is a sectional schematic diagram of a second plate of the splint illustrated in FIG. 1. As illustrated in FIGS. 1-4, a splint 100 comprises a plurality of plates 110 and a splint adjustment device 120.

The plurality of plates 110 of the splint 100 comprise a first plate 111, a second plate 112, a third plate 113, and a fourth plate 114. For example, the first plate 111 and the second plate 112 are oppositely disposed, and the third plate 113 and the fourth plate 114 are oppositely disposed. A space surrounded by the plurality of plates 110 can accommodate a user's limb, such as a patient's injured limb, e.g., a leg, an arm, or the like, so as to fix and protect the injured limb. For example, each of the plates can be made of a polymer material, such as a glass fiber, a polyurethane, a resin, or the like, and other suitable materials may also be used. The embodiments of the present disclosure are not limited in this aspect.

It should be noted that, in the embodiments of the present disclosure, the number of the plurality of plates 110 is not limited to four in the case described above, and may also be other suitable values, such as 2-6, and the embodiments of the present disclosure are not limited thereto. Sizes of the plurality of plates 110 are not limited, and can be determined according to the size of the user's limb or empirical values. The sizes of the plurality of plates 110 can be the same or different. For example, in some example, the size of the first plate 111 and the size of the second plate 112 are the same, the size of the third plate 113 and the size of the fourth plate 114 are the same, and the size of the first plate 111 and the second plate 112 is greater than the size of the third plate 113 and the fourth plate 114. In this way, the plurality of plates 110 can better fit the user's limb, thereby providing better functions of fixing, protection, and so on.

For example, the splint adjustment device 120 is connected to the plurality of plates 110 and configured to allow the plurality of plates 110 to move relatively close to each other or relatively away from each other. For example, the splint adjustment device 120 comprises a drawstring 121 and a drawstring drive device 122.

The drawstring 121 is movably connected to the plurality of plates 110. For example, a plurality of through holes are provided at edges of the first plate 111, the second plate 112, the third plate 113, and the fourth plate 114, and the drawstring 121 passes through the through holes of each plate, and thus is movably connected to the plurality of plates 110. For example, the drawstring 121 passes through the plurality of through holes in an S-shaped cross manner or other suitable manners, different manners may have different effects on a length of the drawstring 121, the fastening degree of the connection between the plates 110, and so on, and the manners may be determined according to actual needs.

The drawstring drive device 122 is disposed on one of the plurality of plates 110, for example, disposed on the first plate 111. The drawstring drive device 122 is connected to the drawstring 121 and is configured to be controllable to drive the drawstring 121, so that the drawstring 121 can be tightened or relaxed under a driving function of the drawstring drive device 122, so as to drive the plurality of plates 110 to be relatively close to each other or relatively away from each other. In a case where the drawstring 121 is relaxed to allow the plurality of plates 110 to move relatively away from each other, the space surrounded by the plurality of plates 110 becomes larger; and in a case where the drawstring 121 is tightened to allow the plurality of plates 110 to move relatively close to each other, the space surrounded by the plurality of plates 110 becomes smaller. Therefore, by tightening or relaxing the drawstring 121 using the drawstring drive device 122, the space surrounded by the plurality of plates 110 can become larger or smaller according to user's requirements. In a case where the splint 100 is used for different users and for different limbs, the splint 100 can be attached to the limb well, thereby realizing the functions of fixing, protection, and so on.

It should be noted that, in the embodiments of the present disclosure, the specific implementation manner of the drawstring drive device 122 is not limited. For example, in some embodiments, the drawstring drive device 122 can be implemented in a form of a combination of a knob, a drawstring reel slot, and a fixed shaft, thereby reducing the cost. For example, in other embodiments, the drawstring drive device 122 can be implemented in a form of a combination of a motor, a motor mount, and a drawstring reel slot, thereby improving adjustment efficiency and saving time and effort.

For example, at least one of the plurality of plates 110 comprises at least two split portions, and the at least two split portions are configured to be separated from each other or be combined with each other, so as to allow the space surrounded by the plurality of plates 110 to be opened or closed. For example, in some examples, as illustrated in FIGS. 1-4, the second plate 112 comprises at least two split portions, such as a first split portion 1121 and a second split portion 1122. The first split portion 1121 and the second split portion 1122 are configured to be separated from each other or be combined with each other, so that the splint 100 can allow the space surrounded by the plurality of plates 110 to be opened or closed, that is, the splint 100 can be opened or closed at the side surface.

Therefore, in a case where the user uses the splint 100, the first split portion 1121 and the second split portion 1122 can be separated, and the splint 100 can be opened at the side surface, so as to allow the limb of the user to get into the space surrounded by the plurality of plates 110 from the side surface of the splint 100. And then, the first split portion 1121 and the second split portion 1122 are combined, so as to close the space surrounded by the plurality of plates 110. By adjusting the drawstring drive device 122, the drawstring 121 is tightened, so that the plurality of plates 110 are attached to the limb, thereby achieving functions of fixing, protection, and so on.

In this way, the user is not required to stretch or remove the limb to/from the space surrounded by the plurality of plates 110 from an end of the splint 100, and can move the limb to get into or remove out of the space surrounded by the plurality of plates 110 from the side surface of the splint 100, thereby facilitating the operation, avoiding secondary injury to the user, enabling the user to simply and quickly adjust and fix, reducing the workload of the user, improving the work efficiency, and improving the user's experience. Moreover, the splint 100 is formed to an integrated structure rather than a plurality of separate components. Compared to the conventional separate splints, the splint 100 is easy to be stored when not in use, and it is not easy to lose the components of the splint 100.

It should be noted that, in the embodiments of the present disclosure, any one or more plates may be provided with at least two split portions, so that the splint 100 has one or more opening and closing positions, so as to be applicable to different limbs. The number of the split portions of each of the plates comprising the split portions is not limited, and may be two, three or any other values to suit different combinations of the split portions, and the embodiments of the present disclosure are not limited thereto.

FIG. 4 is a sectional schematic diagram of a second plate 112, that is, FIG. 4 is a partial enlarged view of the region A as illustrated in FIG. 3. As illustrated in FIG. 4, the first split portion 1121 comprises a first connecting structure 1123, and the second split portion 1122 comprises a second connecting structure 1124. The first connecting structure 1123 is configured to be combined with the second connecting structure 1124, so as to fix a relative position between the first split portion 1121 and the second split portion 1122. For example, in some examples, the first connecting structure 1123 comprises a buckle and the second connecting structure 1124 comprises a clamping slot, alternatively, the first connecting structure 1123 comprises a clamping slot and the second connecting structure 1124 comprises a buckle. The first connecting structure 1123 and the second connecting structure 1124 can be combined in snap-fitting manner, so that the lateral pulling force can be resisted. It should be noted that, in the embodiments of the present disclosure, the first connecting structure 1123 and the second connecting structure 1124 are not limited to the form of the buckle and the clamping slot, and can be other suitable forms as long as the relative position between the first split portion 1121 and the second split portion 1122 can be fixed, and the embodiments of the present disclosure are not limited thereto.

For example, the first connecting structure 1123 can be integrally formed with the first split portion 1121, and the second connecting structure 1124 can be integrally formed with the second split portion 1122. The first connecting structure 1123 and the second connecting structure 1124 can adopt the same materials as those of the first split portion 1121 and the second split portion 1122, such as polymer materials, thereby simplifying production processes and reducing production costs. Of course, the embodiments of the present disclosure are not limited thereto. In some embodiments, the first connecting structure 1123 and the second connecting structure 1124 may also be made of permanent magnets or electromagnets, and the first connecting structure 1123 and the second connecting structure 1124 are attracted to each other, so that the combination of the first split portion 1121 and the second split portion 1122 is more secure.

For example, as illustrated in FIGS. 2-4, in order to allow the combination of the first split portion 1121 and the second split portion 1122 to be more secure, the second plate 112 (that is, the plate comprising at least two split portions) may further comprises at least one group of adsorption components 130, such as a plurality of groups of adsorption components 130. At least one group of the plurality of groups of adsorption components 130 comprises a plurality of adsorption units 131 and 132, and the plurality of adsorption units 131 and 132 are respectively disposed on each of the at least two split portions, that is, respectively disposed on the first split portion 1121 and the second split portion 1122. The adsorption component 130 is configured to combine the at least two split portions (for example, the first split portion 1121 and the second slit portion 1122) with each other.

For example, the adsorption units 131 and 132 may be permanent magnets or electromagnets, and the adsorption unit 131 attract the adsorption unit 132, so that the first split portion 1121 and the second split portion 1122 are tightly combined by the attraction force between the adsorption unit 131 and the adsorption unit 132, and the first split portion 1121 and the second split portion 1122 are not easy to be separated. Of course, the embodiments of the present disclosure are not limited thereto, and the adsorption units 131 and 132 may also be any suitable components such as Velcro straps, as long as the combination of the first split portion 1121 and the second split portion 1122 can be more secure, and the embodiments of the present disclosure are not limited thereto.

It should be noted that, in the embodiments of the present disclosure, the number of the adsorption components 130 is not limited, and may be any values such as one group, two groups, three groups, etc., which may be determined according to the required securing degree of the combination of the first split portion 1121 and the second split portion 1122. The more the adsorbing components 130 are, the more secure the combination of the first split portion 1121 and the second split portion 1122 is. Shapes of the adsorption units 131 and 132 are not limited, and may be any suitable shapes such as rectangular blocks, circular blocks, or the like. The arrangements of the adsorption units 131 and 132 are also not limited, and for example, the adsorption units 131 and 132 may be respectively attached to the surfaces, which are in contact with each other, of the first split portion 1121 and the second split portion 1122, or may be respectively embedded in the plates of the first split portion 1121 and the second split portion 1122, or may also be other suitable arrangements.

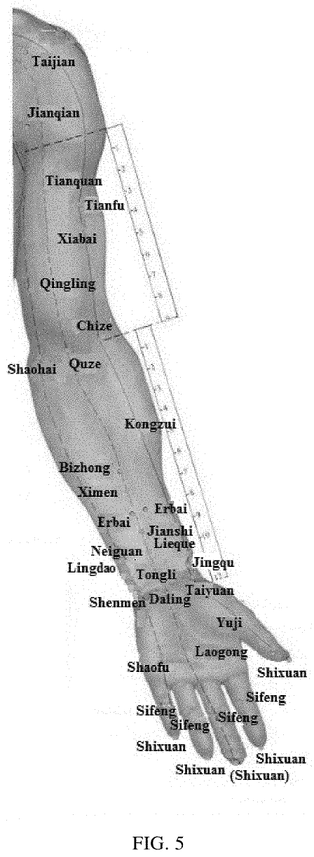

For example, in some embodiments of the present disclosure, in a case where the adsorption units 131 and 132 comprise permanent magnets or electromagnets, the adsorption units 131 and 132 may also be disposed at predetermined positions of the first split portion 1121 and the second split portion 1122 according to a first predetermined pattern. For example, the adsorption units 131 and 132 may be disposed at positions of the first split portion 1121 and the second split portion 1122 corresponding to acupuncture points according to the acupuncture point distribution map of the arm as illustrated in FIG. 5. For example, these acupuncture points may be Lieque, Jianshi, Erbai, Ximen, Bizhong, Kongzui, and the like, and may be any other suitable acupuncture points, and the embodiments of the present disclosure are not limited thereto. Thereby, the adsorption units 131 and 132 can not only allow the combination of the first split portion 1121 and the second split portion 1122 to be more secure, but also provide a magnetic field which can act on the user's acupuncture points.

In some embodiments of the present disclosure, for example, the splint 100 further comprises a pulling force detection circuit 140. The pulling force detection circuit 140 is coupled to the drawstring 121 and is configured to detect a pulling force applied to the drawstring 121. For example, the pulling force detection circuit 140 may be disposed on any one of the plates, for example, disposed on the first plate 111. In order to cooperate with the pulling force detection circuit 140 to realize the pulling force detection function, the drawstring 121 may adopt a structure in which an insulating sheath wraps a core wire. The cross section of the core wire can be deformed under the pulling force, so that the resistance value of the core wire changes. The pulling force detection circuit 140 detects the resistance value of the core wire, and obtains a corresponding pulling force value by analyzing and calculating. For example, the specific relationship between the change value of the resistance and the deformation degree of the core wire, and the specific relationship between the deformation degree and the pulling force value can be obtained by theoretical calculation or test. For example, the material of the core wire may be a piezoelectric ceramic fiber composite material, a conductive rubber, or other suitable materials.

It should be noted that, in the embodiments of the present disclosure, the pulling force detection circuit 140 can be used to detect a resistance value, a capacitance value, or any other parameters, which may be determined according to the structure of the drawstring 121, and the embodiments of the present disclosure are not limit thereto. The pulling force detection circuit 140 can be implemented as a dedicated or general-purpose electronic hardware (or circuit), such as a conventional resistance detection circuit, a capacitance detection circuit, or the like, which is not limited in the embodiments of the present disclosure. For example, the pulling force detection circuit 140 may further comprise a calculation and processing circuit (for example, a processor) for calculating the corresponding pulling force value according to the detected parameters such as resistance values, capacitance values, or the like. The specific configuration of the above electronic hardware is not limited, and may comprise an analog device, a digital chip, or other suitable devices.

In some embodiments of the present disclosure, for example, the splint 100 further comprises a display 150. The display 150 is disposed on one of the plurality of plates 110, for example, on the first plate 111. The display 150 is in signal connection with the pulling force detection circuit 140 and is configured to display a pulling force detection result of the pulling force detection circuit 140. For example, the pulling force detection result comprises one or more of parameters such as a pulling force value, a recommended pulling force range, an indication of tightness degree, and so on. The display 150 receives the pulling force detection result transmitted from the pulling force detection circuit 140 in a wire manner or a wireless manner, and displays in an appropriate manner.

For example, the display 150 can be a display screen, a pointer instrument panel (such as a mechanical meter or a digital tube), or other suitable display devices, which is not limited in the embodiments of the present disclosure. The display screen is, for example, an organic light emitting diode (OLED) display screen, a liquid crystal display (LCD) screen, an electronic paper display screen, or the like. The size of the display 150 is not limited and can be determined according to actual needs, for example, according to factors such as the size of the plate, the amount of display content, and so on.

By using the pulling force detection circuit 140 and the display 150, the splint 100 can facilitate the user to monitor the tightness degree, help the user to adjust the appropriate tightness degree, and prevent the looseness of the splint 100, thereby improving the user's experience.

For example, as illustrated in FIGS. 1-3, the drawstring drive device 122, the pulling force detection circuit 140, and the display 150 are disposed on a plate opposite to the plate comprising the at least two split portions (for example, the second plate 112), that is, disposed on the first plate 111, so as to prevent the magnetism of the adsorption units 131 and 132 in the second plate 112 from affecting the normal operations of the drawstring drive device 122, the pulling force detection circuit 140, and the display 150. Of course, the embodiments of the present disclosure are not limited thereto, the drawstring drive device 122, the pulling force detection circuit 140, and the display 150 may also be disposed on any suitable plate, and the three may be disposed on the same plate or different plates. The embodiments of the present disclosure are not limited in this aspect.

It should be noted that, in some embodiments of the present disclosure, the splint 100 may comprise more or less components, which may be determined according to actual needs, and the embodiments of the present disclosure are not limited thereto. For example, in some embodiments, the pulling force detection circuit 140 and the display 150 may be omitted, and the splint 100 only comprises the plurality of plates 110 and the splint adjustment device 120. For example, in other embodiments, the splint 100 may further comprise a reminder device (such as a buzzer) which is configured to send a reminder to the user, for example, an alarm sound, in a case where the splint is loose, that is, in a case where the pulling force value detected by the pulling force detection circuit 140 is less than a preset value. For example, in still other embodiments, the splint 100 can further comprise a power source, and the power source is configured to provide power to the pulling force detection circuit 140 and the display 150, and in the case where the splint adjustment device 120 comprises a motor, the motor can also be powered.

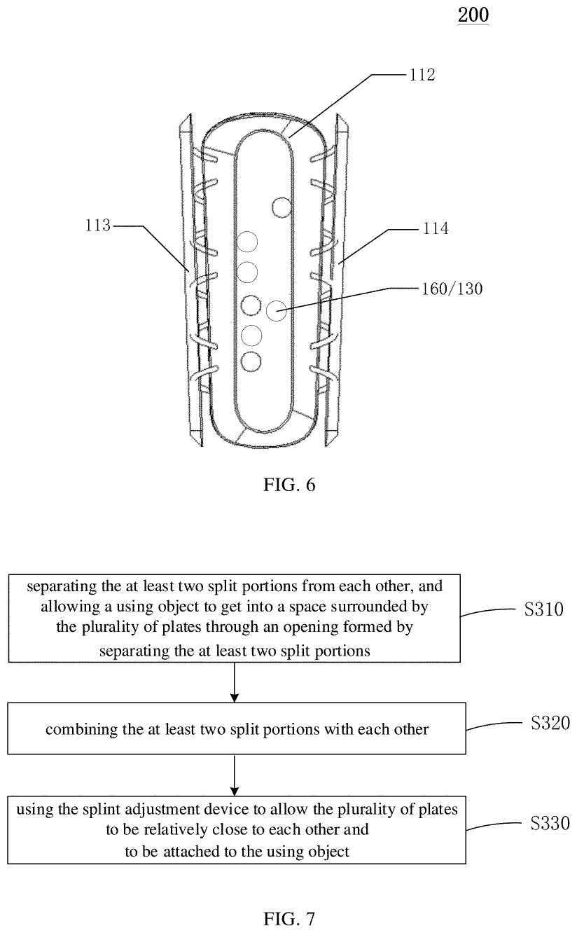

FIG. 6 is a back view of another splint according to an embodiment of the present disclosure. As illustrated in FIG. 6, a splint 200 provided by this embodiment is substantially the same as the splint 100 illustrated in FIGS. 1-4, except for further comprising at least one magnetic component 160. It should be noted that, in the splint 200, the second plate 112 also comprises at least two split portions. In order to clearly illustrate the magnetic component 160, structures of the split portions of the second plate 112 is not explicitly illustrated in FIG. 6, which does not constitute a limitation to the embodiments of the present disclosure.

For example, the magnetic component 160 is disposed on at least one of the plurality of plates 110 according to a second predetermined pattern, for example, disposed on the second plate 112. For example, in some examples, the second predetermined pattern is the same as the first predetermined pattern described above. For example, the magnetic component 160 may be disposed at positions of the second plate 112 corresponding to acupuncture points according to the acupuncture point distribution map of the arm as illustrated in FIG. 5. For example, these acupuncture points may be Lieque, Jianshi, Erbai, Ximen, Bizhong, Kongzui, or the like, and may be any other suitable acupuncture points, and the embodiments of the present disclosure are not limited thereto.

For example, the magnetic component 160 can be a permanent magnet or an electromagnet, and the magnetic component 160 is configured to provide a magnetic field which can act on acupuncture points of the user. The number of the magnetic components 160 is not limited and may be one or more, which can be determined according to the number of acupuncture points that need to be acted on. The shape and the size of the magnetic component 160 are also not limited, and may be determined, for example, according to the effect on the acupuncture points. The splint 200 can provide a magnetic field which can act on the user's acupuncture points, thereby improving the user's experience.

It should be noted that the magnetic component 160 can be a separately provided component, or may be the adsorption units 131 and 132 of the adsorption component 130. In a case where the magnetic component 160 is a separately provided component, the adsorption units 131 and 132 may adopt Velcro straps or other non-magnetic components, thereby reducing the requirements on the materials of the adsorption units 131 and 132. In a case where the magnetic components 160 are the adsorption units 131 and 132 of the adsorption component 130, that is, in a case where the adsorption units 131 and 132 function as the magnetic components 160 and no other magnetic components are separately provided, the number of the components in the splint 200 can be reduced.

At least one embodiment of the present disclosure provides an operation method of the splint provided by any one of the embodiments of the present disclosure. The operation method can be used to operate the splint provided by the embodiments of the present disclosure, and can open or close the splint at the side surface, which is convenient for operation, and can enable the user to easily and quickly adjust and fix the splint, thereby reducing the workload of the user, improving the work efficiency, and improving the use's experience. The splint provided by at least some of the embodiments can also monitor the tightness degree.

FIG. 7 is a flow diagram of an operation method of a splint provided by some embodiments of the present disclosure. For example, in some examples, as illustrated in FIG. 7, the operation method of the splint comprises following operations.

Step S310: separating the at least two split portions from each other, and allowing a using object to get into a space surrounded by the plurality of plates 110 through an opening formed by separating the at least two split portions;

Step S320: combining the at least two split portions with each other; and

Step S330: using the splint adjustment device 120 to allow the plurality of plates 110 to be relatively close to each other and to be attached to the using object.

For example, the at least two split portions comprise a first split portion 1121 and a second split portion 1122. For example, the using object is a limb of the user, such as an injured limb of the patient, e.g., a leg, an arm, or the like.

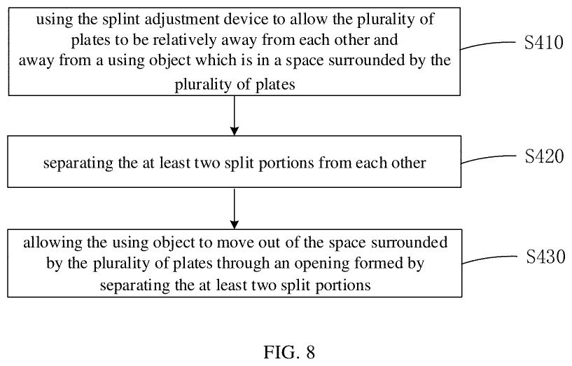

FIG. 8 is a flow diagram of another operation method of a splint provided by some embodiments of the present disclosure. For example, in some examples, as illustrated in FIG. 8, the operation method of the splint comprises following operations.

Step S410: using the splint adjustment device 120 to allow the plurality of plates 110 to be relatively away from each other and away from a using object which is in a space surrounded by the plurality of plates 110;

Step S420: separating the at least two split portions from each other; and

Step S430: allowing the using object to move out of the space surrounded by the plurality of plates 110 through an opening formed by separating the at least two split portions.

For detail contents and technical effects of the above operation methods, reference can be made to the above descriptions of the splint 100/200, and details are not described here again.

The following several statements should be noted.

(1) The accompanying drawings involve only the structure(s) in connection with the embodiment(s) of the present disclosure, and other structure(s) can be referred to common design(s).

(2) In case of no conflict, embodiments of the present disclosure and the features in the embodiments may be mutually combined to obtain new embodiments.

The above descriptions are only specific embodiments of the present disclosure, but the protection scope of the present disclosure is not limited thereto, the protection scope of the present disclosure should be determined by the protection scope of the claims.

What is claimed is:

1. A splint, comprising:
a plurality of plates; and
a splint adjustment device connected to the plurality of plates and configured to allow the plurality of plates to move relatively close to each other or relatively away from each other,
wherein at least one of the plurality of plates comprises at least two split portions, and the at least two split portions are configured to be separated from each other or be combined with each other, so as to allow a space surrounded by the plurality of plates to be opened or closed.

2. The splint according to claim 1, wherein the at least two split portions comprise a first split portion and a second split portion,
the first split portion comprises a first connecting structure, and the second split portion comprises a second connecting structure, and
the first connecting structure is configured to be combined with the second connecting structure, so as to fix a relative position between the first split portion and the second split portion.

3. The splint according to claim 2, wherein the first connecting structure comprises a buckle and the second connecting structure comprises a clamping slot, alternatively,
the first connecting structure comprises a clamping slot and the second connecting structure comprises a buckle.

4. The splint according to claim 3, wherein a material of the first connecting structure or a material of the second connecting structure comprises a permanent magnet or an electromagnet.

5. The splint according to claim 1, wherein at least one of the plurality of plates comprising the at least two split portions comprises at least one group of adsorption components,
the at least one group of adsorption components comprises a plurality of adsorption units, the plurality of adsorption units are respectively on each of the at least two split portions, and the adsorption components are configured to enable the at least two split portions to combine with each other.

6. The splint according to claim 5, wherein the adsorption units comprise permanent magnets, electromagnets, or Velcro straps.

7. The splint according to claim 5, wherein the adsorption units comprise permanent magnets or electromagnets, and
the adsorption units are disposed at predetermined positions of the split portions according to a first predetermined pattern.

8. The splint according to claim 1, wherein the splint adjustment device comprises:
a drawstring movably connected to the plurality of plates; and
a drawstring drive device on one of the plurality of plates, connected to the drawstring and configured to be controllable to drive the drawstring, so that the drawstring is capable of being tightened or relaxed under a driving function of the drawstring drive device, to drive the plurality of plates to be relatively close to each other or relatively away from each other.

9. The splint according to claim 8, further comprising a pulling force detection circuit,
wherein the pulling force detection circuit is coupled to the drawstring and is configured to detect a pulling force applied to the drawstring.

10. The splint according to claim 9, further comprising a display,
wherein the display is on one of the plurality of plates, is in signal connection with the pulling force detection circuit, and is configured to display a pulling force detection result of the pulling force detection circuit.

11. The splint according to claim 10, wherein the display and the drawstring drive device are on a plate opposite to a plate comprising the at least two split portions.

12. The splint according to claim 1, further comprising at least one magnetic component,
wherein the at least one magnetic component is disposed on at least one plate of the plurality of plates according to a second predetermined pattern.

13. The splint according to claim 12, wherein the at least one magnetic component comprises a permanent magnet or an electromagnet.

14. An operation method of the splint according to claim 1, comprising:
separating the at least two split portions from each other, and allowing a using object to get into a space surrounded by the plurality of plates through an opening formed by separating the at least two split portions;
combining the at least two split portions with each other; and
using the splint adjustment device to allow the plurality of plates to be relatively close to each other and to be attached to the using object.

15. An operation method of the splint according to claim 1, comprising:
using the splint adjustment device to allow the plurality of plates to be relatively away from each other and away from a using object which is in a space surrounded by the plurality of plates;
separating the at least two split portions from each other; and
allowing the using object to move out of the space surrounded by the plurality of plates through an opening formed by separating the at least two split portions.
